(19) United States
(12) Patent Application Publication
(10) Pub. No.: US 2026/0151523 A1
(43) Pub. Date: Jun. 4, 2026

(54) UV STERILIZER

(71) Applicant: Young Eun HAN, Seoul (KR)

(72) Inventor: Young Eun HAN, Seoul (KR)

(21) Appl. No.: 19/023,542

(22) Filed: Jan. 16, 2025

(30) Foreign Application Priority Data
Dec. 2, 2024 (KR) 10-2024-0176180

(51) Int. Cl. A61L 2/10; A61L 2/24; A61L 2/28; G01J 1/42

(52) U.S. Cl. CPC A61L 2/10; A61L 2/24; A61L 2/28; G01J 1/4204; A61L 2202/11; A61L 2202/14

(57) ABSTRACT

A UV sterilizer that is easy to carry and of which an installation position can be freely changed can include a housing having an inner space; a UV lamp installed in the inner space and emitting UV light; a battery installed in the inner space and providing operating power to the UV lamp; an illuminance sensor installed in the housing and detecting a brightness of an environment outside the housing; and a main printed circuit board (PCB) installed in the inner space and on which a control processor controlling an operation of the UV lamp based on a detection signal of the illuminance sensor is installed.

### BACKGROUND

### 1. Field of the Invention

The present disclosure relates to an ultraviolet (UV) sterilizer, and more particularly, to a UV sterilizer that is easy to carry, stored in a suitcase or a wardrobe, and capable of sterilizing clothes.

### 2. Discussion of Related Art

When residing in an outside environment such as when traveling for a long period of time away from home, e.g., abroad, there is a risk of infection, and it is difficult to use medical services.

In particular, there is a high risk of contracting an infectious disease when exposed to bacteria and viruses in various places, but even in this case, it is not easy to wash clothes abroad, and thus there is a need for a portable device that can sterilize clothes that one has worn.

Korean Unexamined Patent Application Publication No. 10-2024-0068290 discloses a suitcase having a UV sterilizer installed therein. However, for the UV sterilizer installed inside the suitcase, a sterilization place is limited to the inside of the corresponding suitcase. In addition, there are inconveniences that when a battery, which is a power source for operating the sterilizer, is configured integrally with the suitcase, the suitcase cannot be carried as hand baggage on a plane, and when the battery is removed, an external power source should be used.

In addition, although Korean Unexamined Utility Model Application Publication No. 20-2023-0001365 and Korean Unexamined Patent Application Publication No. 10-2022-0150040 disclose a pen type portable UV sterilizer, the pen type UV sterilizer requires a manual operation by a user for starting and stopping sterilization and makes it necessary for a user to directly radiate UV light to an object to be sterilized while holding the pen type UV sterilizer by hand, and thus the pen type UV sterilizer may be cumbersome to use, and when the pen type UV sterilizer is manually operated, there is a risk of exposure of the user's skin to UV light.

### RELATED ART DOCUMENTS

### Patent Documents

Related Art Document 1: Korean Unexamined Patent Application Publication No. 10-2024-0068290

Related Art Document 2: Korean Unexamined Utility Model Application Publication No. 20-2023-0001365

Related Art Document 3: Korean Unexamined Patent Application Publication No. 10-2022-0150040

### SUMMARY OF THE INVENTION

The present disclosure has been devised to address at least some of the problems of the related art mentioned above, and one aspect of the present disclosure is directed to providing a UV sterilizer that is easy to carry and of which an installation position can be freely changed.

In addition, another aspect of the present disclosure is directed to providing a UV sterilizer of which a sterilization operation can start and stop automatically and that can fundamentally prevent exposure of the skin of a user to UV light for sterilization.

One embodiment of the present disclosure provides a UV sterilizer including a housing having an inner space; a UV lamp installed in the inner space and emitting UV light; a battery installed in the inner space and providing operating power to the UV lamp; an illuminance sensor installed in the housing and detecting a brightness of an environment outside the housing; and a main printed circuit board (PCB) installed in the inner space and on which a control processor controlling an operation of the UV lamp based on a detection signal of the illuminance sensor is installed.

In one embodiment, the control processor may be configured to turn on the UV lamp in a case in which the detection signal of the illuminance sensor indicates that the environment outside the housing is dark and may be configured to turn off or not turn on the UV lamp in a case in which the detection signal of the illuminance sensor indicates that the environment outside the housing is bright.

In addition, in one embodiment, the control processor may be configured to turn off the UV lamp regardless of the detection signal of the illuminance sensor when a predetermined sterilization time passes from a time the UV lamp is turned on.

In addition, in one embodiment, a speaker transmitting sound to the outside of the housing may be further provided in the inner space, and the control processor may be configured to transmit voice guidance through the speaker before a start of a UV sterilization operation that is caused by the UV lamp being turned on and after an end of the UV sterilization operation that is caused by the UV lamp being turned off.

In addition, in one embodiment, the control processor may transmit a warning sound through the speaker in a case in which the UV lamp is turned on and the detection signal of the illuminance sensor indicates that the environment outside the housing is bright before the sterilization time passes.

In addition, in one embodiment, the UV lamp may be configured as a plurality of UV-C light emitting diode (LED) elements mounted on a lamp PCB, and the lamp PCB may be configured as a plurality of lamp PCBs to arrange the UV lamps in directions different from each other in the housing.

In addition, in one embodiment, the plurality of lamp PCBs may be configured to receive power independently from each other.

In addition, the UV sterilizer according to one embodiment of the present disclosure may further include a fan member installed in the inner space and introducing air outside the housing into the housing.

Meanwhile, in one embodiment, the illuminance sensor may be configured as a plurality of illuminance sensors disposed in directions different from each other in the housing, and the control processor may be configured to turn on the UV lamp in a case in which detection signals of the plurality of illuminance sensors all indicate that the environment outside the housing is dark and may be configured to turn off the UV lamp in a case in which a detection signal of at least one illuminance sensor among the plurality of illuminance sensors indicates that the environment outside the housing is bright.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a front perspective view of a UV sterilizer according to one embodiment of the present disclosure;

FIG. 2 is a back perspective view of a state in which a lamp cover of the UV sterilizer illustrated in FIG. 1 is removed;

FIG. 3 is a perspective view illustrating an installation structure using a strap of the UV sterilizer illustrated in FIG. 1;

FIG. 4A is a right side view of the state in which the lamp cover of the UV sterilizer illustrated in FIG. 1 is removed, and FIGS. 4B, 4C, and 4D are a front view, a left side view, and a back view, respectively, of the UV sterilizer;

FIG. 5 is an exploded perspective view of the UV sterilizer illustrated in FIG. 1;

FIG. 6 is a cross-sectional view illustrating an internal configuration of the UV sterilizer illustrated in FIG. 1;

FIG. 7 is a view illustrating an installation structure of a fan member of the UV sterilizer illustrated in FIG. 1; and

FIG. 8A is a right side view of a state in which a lamp cover of a UV sterilizer according to another embodiment of the present disclosure is removed, and FIGS. 8B, 8C, and 8D are a front view, a left side view, and a back view, respectively, of the UV sterilizer according to another embodiment of the present disclosure.

### DETAILED DESCRIPTION OF EXEMPLARY EMBODIMENTS

Terms used herein are only used to describe specific embodiments and are not intended to limit the present disclosure. In addition, in the present specification, a singular expression includes a plural expression unless the context clearly indicates otherwise.

Hereinafter, embodiments of the present disclosure will be described with reference to the accompanying drawings.

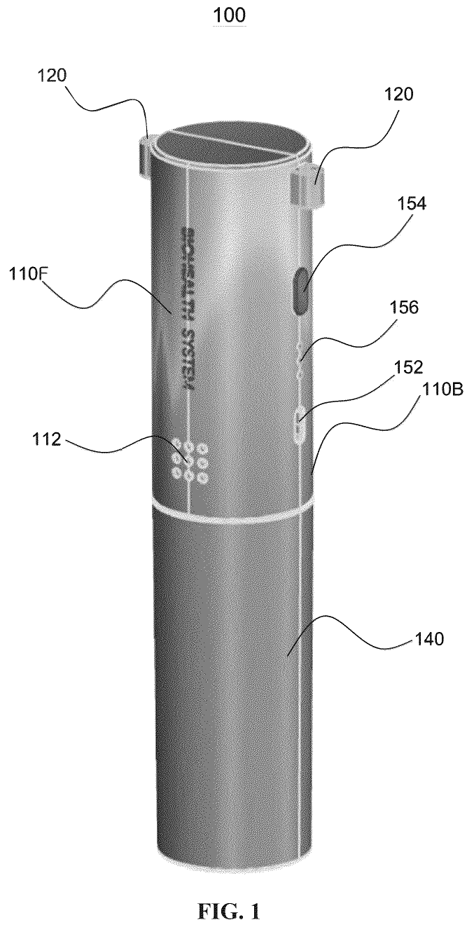

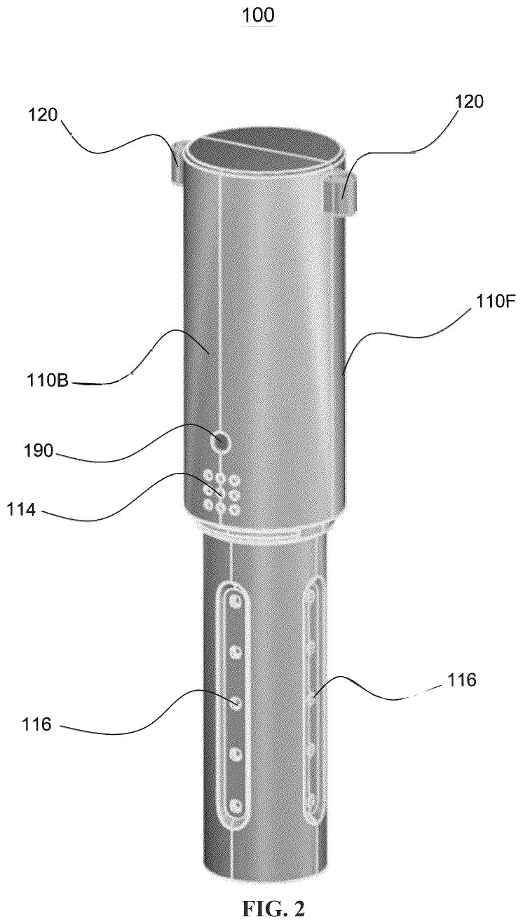

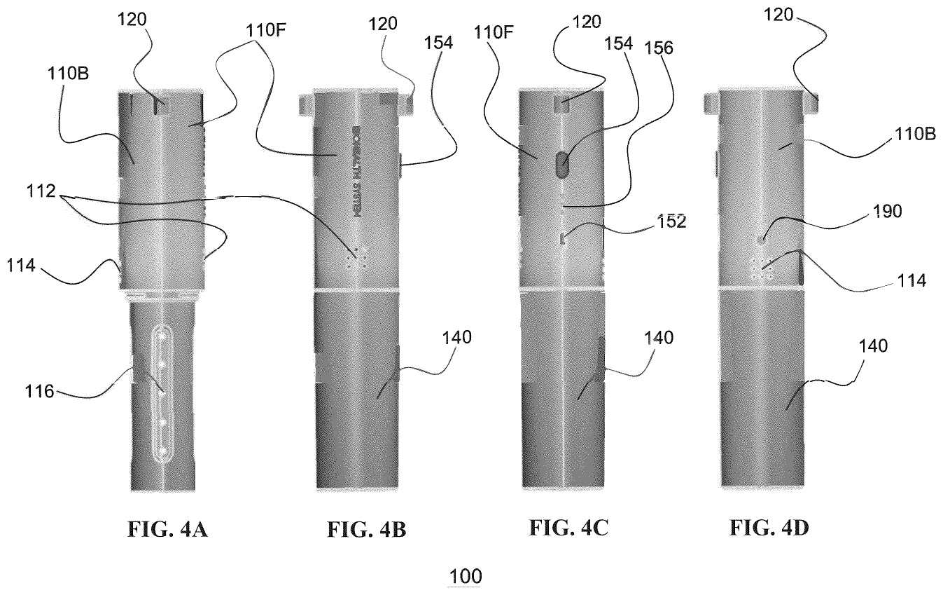

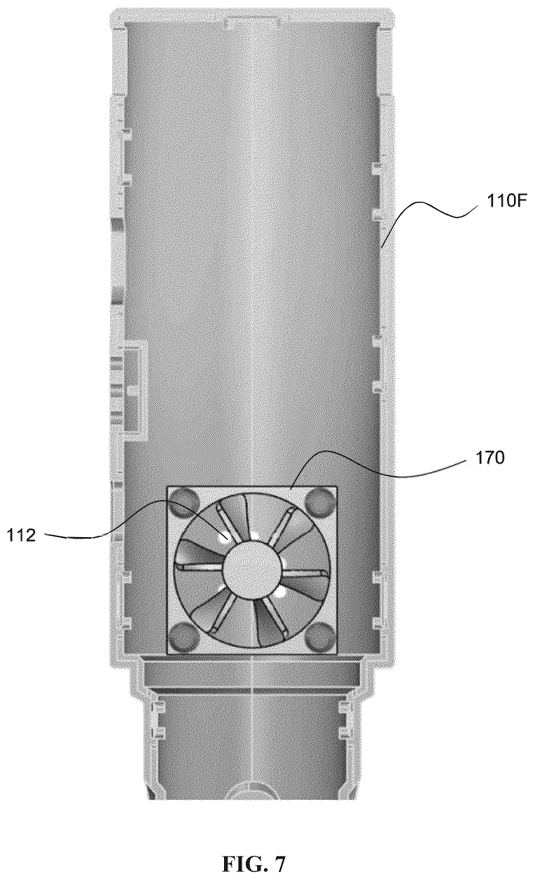

First, a UV sterilizer 100 according to one embodiment of the present disclosure will be described with reference to FIGS. 1 to 7.

As illustrated in FIGS. 1 to 7, the UV sterilizer 100 according to one embodiment of the present disclosure may include a housing, a strap bracket 120, a UV lamp 130, a lamp PCB 135, a lamp cover 140, a PCB bracket 138, a main PCB 150, a battery 160, a power button 154, an indicator 156, a fan member 170, a speaker 180, and an illuminance sensor 190.

The housing may constitute an exterior of the UV sterilizer 100 according to one embodiment of the present disclosure and may have a space in which the UV lamp 130, the lamp PCB 135, the PCB bracket 138, the main PCB 150, the battery 160, the power button 154, the indicator 156, the fan member 170, the speaker 180, and the illuminance sensor 190, which will be described below, are embedded.

In one embodiment, the housing may include a front housing 110F and a back housing 110B assembled to each other and forming the space.

In addition, in one embodiment, the housing may be configured in the form of a bar to minimize the space occupied at the time of installation and to facilitate storage at the time of carrying. For durability against external impact to be relatively high, for example, the housing may be implemented to constitute a cylindrical structure as a whole together with the lamp cover 140, which will be described below, when the housing is mounted on the lamp cover 140, but the present disclosure is not limited thereto, and it is self-evident that the housing may also be configured to constitute any polygonal tubular form.

In one embodiment, the front housing 110F may be divided into an upper section in which the battery 160, the main PCB 150, the fan member 170, and the speaker 180 are accommodated and a lower section in which the UV lamp 130 and the lamp PCB 135 are accommodated.

Here, a lamp hole 116 for outputting UV light generated by the UV lamp 130 to the outside of the front housing 110F may be formed in the lower section of the front housing 110F.

In one embodiment, when the UV lamp 130 is configured as a plurality of UV-C LED lamps, the lamp hole 116 may be provided as a plurality of lamp holes 116 formed to correspond one-to-one to the UV-C LED lamps.

In addition, in one embodiment, a ventilation hole 112 through which outside air may flow into the housing may be provided in the front housing 110F.

In addition, the back housing 110B may have a structure that is symmetrical to the front housing 110F as a whole.

Like the front housing 110F, the back housing 110B may also be divided into an upper section in which the battery 160, the main PCB 150, the fan member 170, and the speaker 180 are accommodated and a lower section in which the UV lamp 130 and the lamp PCB 135 are accommodated.

In addition, the lamp hole 116 described above may also be formed in the lower section of the back housing 110B.

In one embodiment, a speaker hole 114 for outputting sound of the speaker 180, which will be described below, to the outside may be formed in the back housing 110B.

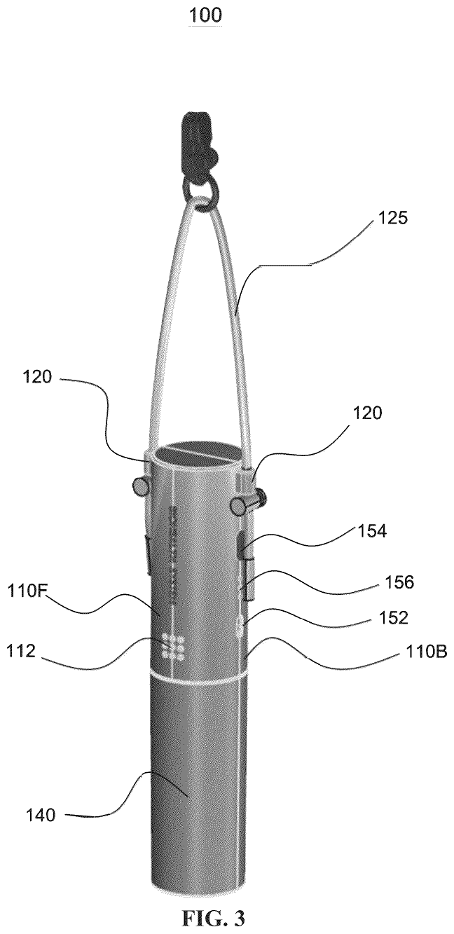

The strap bracket 120 is coupled to the housing, and as illustrated in FIG. 3, constitutes a structure to which a strap 125 can be fastened from outside the housing.

The strap bracket 120 may mediate coupling between the strap 125 and the housing, and the strap 125 may be hung on an object such as a hook inside a suitcase or a wardrobe.

In one embodiment, the strap bracket 120 may be configured so that a length of the strap 125 extending from the strap bracket 120 is adjustable.

A user may adjust the length of the strap 125 fastened to the strap bracket 120 and may install the strap 125 inside a suitcase or a wardrobe.

The UV lamp 130 is a member generating UV light for UV sterilization and may be installed inside the housing.

In one embodiment, the UV lamp 130 may be configured as UV-C LED elements that consume little power and are small in size.

In addition, in one embodiment, the UV lamp 130 may be configured as LED elements that generate UV light having a wavelength of 222 nm in the UV-C wavelength band. Since the UV light having the wavelength of 222 nm is not able to reach the stratum corneum of human skin, there is an advantage that it is safe even when the user's skin is exposed to the UV light.

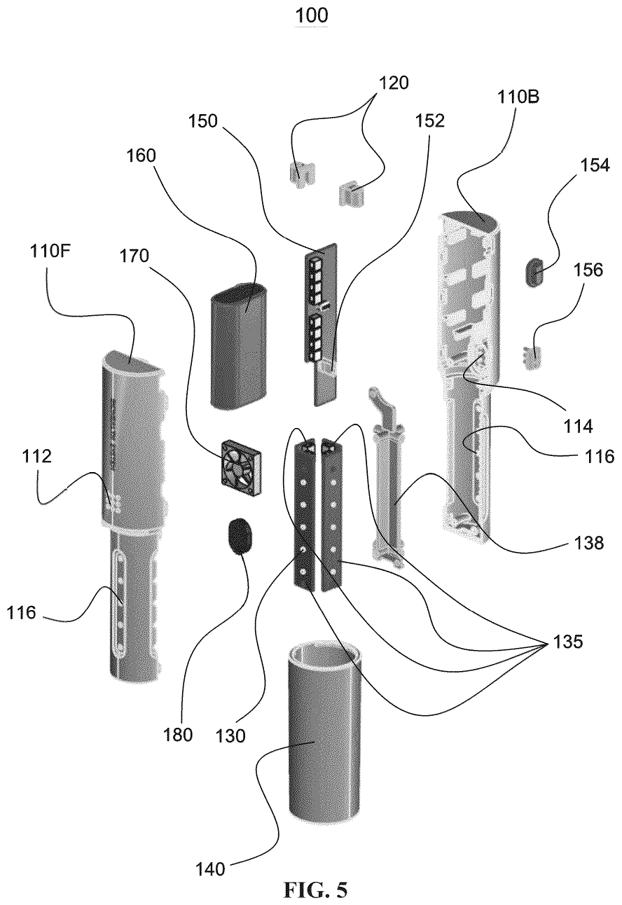

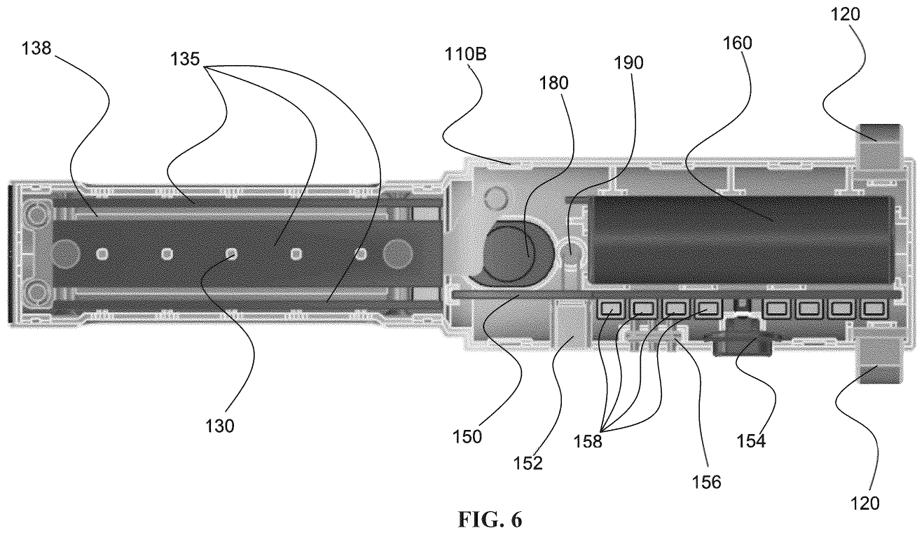

As illustrated in FIGS. 5 and 6, the UV lamp 130 may be configured as a plurality of LED elements, and the plurality of LED elements may be mounted to be arranged at predetermined intervals on the lamp PCB 135.

Here, the lamp PCB 135 may receive power from the battery 160, which will be described below, and may apply power to the plurality of UV lamps 130 mounted thereon.

The UV sterilizer 100 according to one embodiment of the present disclosure may radiate UV light to a wide region through the plurality of UV lamps 130 arranged on a single lamp PCB 135.

In addition, in one embodiment, the lamp PCB 135 may be configured as a plurality of lamp PCBs 135.

Here, as illustrated in FIGS. 5 and 6, the plurality of lamp PCBs 135 may be arranged with different orientations from each other so that the UV lamps 130 are disposed in directions different from each other in the housing.

In one embodiment illustrated in FIG. 5, four lamp PCBs 135 may be installed toward four different sides of the housing. In this way, UV light may be emitted through the four sides of the housing.

Accordingly, since a sterilization operation is possible through UV light emitted through four different sides regardless of a direction in which the UV sterilizer 100 according to one embodiment of the present disclosure is installed, there are advantages in that a degree of freedom of installation is very high, and a sterilization area that is large enough to perform sterilization thoroughly around the housing can be secured.

However, the UV sterilizer 100 according to one embodiment of the present disclosure is not limited to the embodiment in which the UV lamps 130 are disposed at four sides of the housing as described above, and the UV lamps 130 may be disposed in two different directions, three different directions, or five or more different directions, and the UV lamp 130 may be additionally disposed at a lower end of the housing.

Meanwhile, the positions of the plurality of lamp PCBs 135 on which the plurality of UV lamps 130 are mounted may be fixed inside the housing via the PCB bracket 138 as illustrated in FIG. 5.

The design of the PCB bracket 138 may be changed to various forms to correspond to various arrangement forms of the lamp PCBs 135.

The lamp cover 140 is a member configured to cover a lower section of the housing, that is, a member configured to collectively cover the lower section of the front housing 110F and the lower section of the back housing 110B when the front housing 110F and the back housing 110B are assembled to each other.

The lamp cover 140 may serve as a UV transmission window that transmits UV light while covering and protecting the UV lamps 130 exposed to the outside of the housing through the lamp holes 116.

In one embodiment, the lamp cover 140 may be implemented in a transparent color that enables UV light having a wavelength in the UV-C wavelength band to be transmitted with high transmittance and may be made of a polycarbonate (PC) material having excellent rigidity.

The main PCB 150 may be installed in an upper section of the housing as illustrated in FIG. 6 and may be electrically connected to the battery 160, the power button 154, the indicator 156, the fan member 170, the speaker 180, and the illuminance sensor 190 which will be described below.

In addition, the main PCB 150 may be electrically connected to the lamp PCBs 135, may transmit power supplied to the UV lamps 130, and may transmit a control signal of a control processor (not illustrated) for controlling the on/off of the UV lamps 130 to the lamp PCBs 135.

The control processor installed on the main PCB 150 may control the UV lamps 130, the indicator 156, the fan member 170, and the speaker 180 based on an input signal of the power button 154 and a detection signal of the illuminance sensor 190 which will be described below.

The main PCB 150 may be fastened to the back housing 110B as illustrated in FIG. 6.

In addition, in one embodiment, a plurality of power supply sockets 158 that correspond one-to-one to the plurality of lamp PCBs 135 may be provided on the main PCB 150. The plurality of power supply sockets 158 may be controlled independently.

Accordingly, in the UV sterilizer 100 according to one embodiment of the present disclosure, the plurality of UV lamps 130 may be operated independently for each lamp PCB 135 according to settings.

The configuration in which the UV lamps 130 disposed in a plurality of directions can operate independently as described above has an advantage in that, when the UV sterilizer 100 is disposed to be attached to one wall of a suitcase or a wardrobe, unnecessary power consumption can be reduced by setting the UV lamps 130 on the wall side to selectively not operate.

The battery 160 is installed inside the housing and may constitute power used in the UV lamps 130, the fan member 170, the indicator 156, the speaker 180, and the illuminance sensor 190.

The battery 160 may be charged through a charging terminal 152 exposed to the outside of the housing.

The power button 154 is provided to be exposed on a surface of the housing and is configured to allow a user to turn the UV sterilizer 100 according to one embodiment of the present disclosure on or off.

The indicator 156 may be disposed to be exposed to the outside of the housing and may display a remaining capacity of the battery 160.

In addition, in one embodiment, the indicator 156 may display on the outside of the housing a state in which the power of the UV sterilizer 100 is turned on.

The UV sterilizer 100 according to one embodiment of the present disclosure may be configured so that the state in which the power is turned on can be checked through the indicator 156 because whether the UV lamps 130 are turned on cannot be identified through visual inspection.

The fan member 170 may be installed inside the housing and may perform a function of circulating outside air into the housing to prevent overheating of electric parts installed inside the housing.

Also, in addition to performing the air circulating function, the fan member 170 draws air inside a suitcase or a wardrobe toward the housing to move microscopic bacteria or viruses floating in the air to around the UV lamps 130 so that the bacteria or viruses are exposed to UV light, and in this way, sterilization performance inside a sealed space can be improved.

The speaker 180 may be installed at the speaker hole 114 of the back housing 110B and may transmit sound.

In one embodiment, the control processor may transmit voice guidance through the speaker 180 before a start of a UV sterilization operation and after an end of the UV sterilization operation.

For example, the voice guidance may be transmitted in various languages, such as Korean, English, and Chinese, according to set values.

In addition, the control processor may generate a warning sound through the speaker 180 according to a detection signal of the illuminance sensor 190 which will be described below.

The illuminance sensor 190 may be configured to detect brightness of an environment outside the housing.

The illuminance sensor 190 may have a sensing surface disposed in a hole formed in one side of the housing and may detect the brightness of the outside of the housing.

In one embodiment, the illuminance sensor 190 may send a detected value as a detection signal to the control processor.

For safe use of the UV sterilizer 100 according to one embodiment of the present disclosure, the illuminance sensor 190 may operate for a function of allowing UV sterilization to be performed only in a dark environment.

Specifically, when the UV sterilizer 100 according to one embodiment of the present disclosure is turned on in a bright environment, the UV lamps 130 may not be turned on because the detection signal of the illuminance sensor 190 indicates that the environment outside the housing is bright, and in this state, when the environment outside the housing becomes dark due to closing the suitcase, closing the door of the wardrobe, etc., and the detection signal of the illuminance sensor 190 is changed and indicates that the environment outside the housing is dark, the UV lamps 130 may be turned on automatically.

In addition, when a sterilization operation is performed in a state in which the UV lamps 130 are turned on, and then the environment outside the housing becomes bright due to opening the suitcase, opening the door of the wardrobe, etc., and the detection signal of the illuminance sensor 190 is changed and indicates that the environment outside the housing is bright, the UV lamps 130 may be turned off automatically to prevent the user from being exposed to UV light.

Meanwhile, in one embodiment, the control processor may limit a sterilization time to a predetermined length of time from a time the UV lamps 130 are turned on and may set the UV lamps 130 to be turned off automatically as soon as the sterilization time passes, regardless of the detection signal of the illuminance sensor 190.

Adding a timer function to the sterilization operation of the UV lamps 130 as described above can simultaneously improve UV sterilization and virus removal effects and minimize problems such as damage to or discoloration of clothing or items that may occur due to long-time exposure to UV light.

For example, the sterilization time of the UV lamps 130 may be set to 10 minutes that is sufficient for sterilization of and virus removal from clothing and does not cause much damage to the clothing, but the present disclosure is not limited thereto.

Meanwhile, the control processor may operate to transmit a warning sound through the speaker 180 as soon as a signal indicating brightness is detected by the illuminance sensor 190 while the sterilization operation is being performed for the sterilization time.

This is to allow the sterilization operation to continue by informing the user of the fact that the sterilization operation has not ended yet when the user opens the suitcase or wardrobe thinking that the sterilization operation has ended. In this way, the spread of bacteria and viruses to the outside can be prevented fundamentally.

However, when the detection signal of the illuminance sensor 190 continues to indicate brightness and does not indicate darkness again even after the warning sound is transmitted, the control processor may, as described above, turn off the UV lamps 130 to prevent the user from being exposed to UV light.

In addition, the UV sterilizer 100 according to one embodiment of the present disclosure may be configured so that, even when the power of the UV sterilizer 100 is turned on and the detection signal of the illuminance sensor 190 indicates darkness, a start time, at which the UV lamps 130 are turned on and the sterilization operation starts, may be set to after a predetermined time, instead of the UV lamps 130 being turned on and the UV sterilization operation starting immediately.

This is a design that takes into account the time required to close a zipper of a suitcase, etc., or a case in which the user immediately retrieves a forgotten item therefrom, and it is to prevent an unnecessary operation in which the UV lamps 130 are repeatedly turned on or off within a short time in immediate response to a detection signal of the illuminance sensor 190.

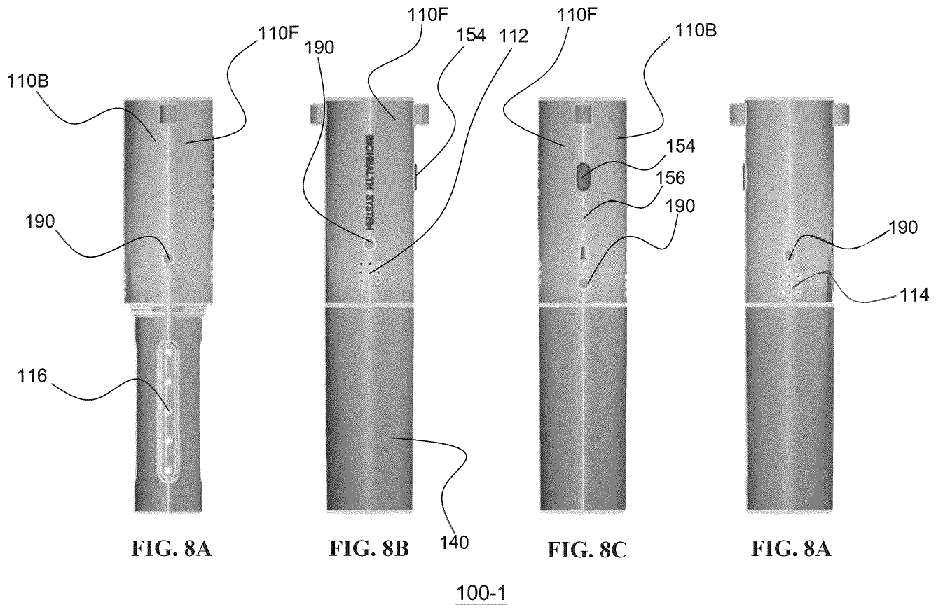

Next, referring to FIG. 8, a UV sterilizer 100-1 according to another embodiment of the present disclosure will be described.

As illustrated in FIG. 8, a configuration in which a plurality of illuminance sensors 190 are provided in various directions in the housing is the only difference of the UV sterilizer 100-1 according to another embodiment of the present disclosure from the UV sterilizer 100 according to one embodiment of the present disclosure that has been described above with reference to FIGS. 1 to 7, and other components of the UV sterilizer 100-1 are substantially the same as those of the above-described UV sterilizer 100 according to one embodiment of the present disclosure.

In another embodiment of the present disclosure, a total of four illuminance sensors 190 may be provided by each illuminance sensor 190 being provided on one of a front surface, a back surface, a left side surface, and a right side surface of a housing.

In such a configuration, a control processor (not illustrated) may perform an operation of turning on UV lamps 130 under a condition that detection signals of the plurality of illuminance sensors 190 disposed in various directions all indicate darkness.

In this way, it is possible to prevent a situation in which, when the illuminance sensors 190 are provided only in one direction of the housing and clothing, an item, or a wall of a suitcase, which is opaque, is disposed to cover the corresponding illuminance sensors 190 in a suitcase or a wardrobe, detection signals of the illuminance sensors 190 may all continue to indicate darkness even when the suitcase is open or the wardrobe is open.

In one embodiment, the control processor may determine that a condition for turning on the UV lamps 130 is satisfied when the detection signals of the plurality of illuminance sensors 190 all indicate darkness, and may determine that a condition for turning off or not turning on the UV lamps 130 is satisfied when a detection signal of at least one illuminance sensor 190 among the plurality of illuminance sensors 190 indicates brightness.

The UV sterilizers 100 and 100-1 according to the embodiments of the present disclosure that have been described above may be installed inside a travel suitcase or a wardrobe and may perform UV sterilization of various articles including clothing to remove sources of infection such as bacteria, severe acute respiratory syndrome (SARS), Coronavirus (COVID), and other viruses and prevent contraction of an infectious disease.

According to a UV sterilizer of one embodiment of the present disclosure having the above configurations, there are advantageous effects that the UV sterilizer can easily be installed at necessary places and can be safely used due to preventing exposure of a user to UV light for sterilization.

Although specific embodiments of the present disclosure have been described above with reference to the drawings, it should be noted that the present disclosure may be modified and changed in various ways by those of ordinary skill in the art within the scope without departing from the spirit and range of the present disclosure stated in the claims below.

What is claimed is:

1. An ultraviolet (UV) sterilizer comprising:
a housing having an inner space;
a UV lamp installed in the inner space and emitting UV light;
a battery installed in the inner space and providing operating power to the UV lamp;
an illuminance sensor installed in the housing and detecting a brightness of an environment outside the housing; and
a main printed circuit board (PCB) installed in the inner space and on which a control processor controlling an operation of the UV lamp based on a detection signal of the illuminance sensor is installed.

2. The UV sterilizer of claim 1, wherein the control processor is configured to turn on the UV lamp in a case in which the detection signal of the illuminance sensor indicates that the environment outside the housing is dark and is configured to turn off or not turn on the UV lamp in a case in which the detection signal of the illuminance sensor indicates that the environment outside the housing is bright.

3. The UV sterilizer of claim 2, wherein the control processor is configured to turn off the UV lamp regardless of the detection signal of the illuminance sensor when a predetermined sterilization time passes from a time the UV lamp is turned on.

4. The UV sterilizer of claim 3, wherein:
a speaker transmitting sound to the outside of the housing is further provided in the inner space; and
the control processor is configured to transmit voice guidance through the speaker before a start of a UV sterilization operation that is caused by the UV lamp being turned on and after an end of the UV sterilization operation that is caused by the UV lamp being turned off.

5. The UV sterilizer of claim 4, wherein the control processor transmits a warning sound through the speaker in a case in which the UV lamp is turned on and the detection signal of the illuminance sensor indicates that the environment outside the housing is bright before the sterilization time passes.

6. The UV sterilizer of claim 1, wherein:
the UV lamp is configured as a plurality of UV-C light emitting diode (LED) elements mounted on a lamp PCB; and
the lamp PCB is configured as a plurality of lamp PCBs to arrange the UV lamps in directions different from each other in the housing.

7. The UV sterilizer of claim 6, wherein the plurality of lamp PCBs are configured to receive power independently from each other.

8. The UV sterilizer of claim 1, further comprising a fan member installed in the inner space and introducing air outside the housing into the housing.

9. The UV sterilizer of claim 1, wherein:
the illuminance sensor is configured as a plurality of illuminance sensors disposed in directions different from each other in the housing; and
the control processor is configured to turn on the UV lamp in a case in which detection signals of the plurality of illuminance sensors all indicate that the environment outside the housing is dark and is configured to turn off the UV lamp in a case in which a detection signal of at least one illuminance sensor among the plurality of illuminance sensors indicates that the environment outside the housing is bright.
